(19) United States
(12) Patent Application Publication
(10) Pub. No.: US 2008/0051688 A1
(43) Pub. Date: Feb. 28, 2008

(54) Two part wound dressing

(76) Inventor: Richard D. Lowe, Brea, CA (US)

Correspondence Address: SANDRA LEE LIPKIN;A LAW CORPORATION, 1891 GOODYEAR AVE., SUITE 622, VENTURA, CA, 93003, US

(21) Appl. No.: 11/890,190

(22) Filed: Aug. 2, 2007

Related U.S. Application Data
(60) Provisional application No. 60/840,412, filed on Aug. 28, 2006.

(51) Int. Cl. A61F 13/02

(52) U.S. Cl. 602/58; 604/307

(57) ABSTRACT

A wound dressing that includes two layers for a two part solution to the needs of a healing skin wound. The first step involves a relatively closed system wherein moisture is kept near the skin to promote healing. The second step allows for removal of a top layer of the dressing to reveal a layer with a series of apertures that allows for 100% moisture vapor and oxygen transmission rates to avoid irritation and itching of the skin. At the same time, the bottom layer of the wound dressing maintains a significant outer perimeter that prevents the perimeter migration of microorganisms into the healing wound. The wound dressing can also be used as a means for securing medical instruments, such as intravenous catheters, to the skin of a patient while avoiding the itching and irritation associated with traditional methods.

### REFERENCE TO PRIOR APPLICATION

This application claims the priority of provisional application 60/840,412, filed Aug. 28, 2006 entitled DUAL-AID/I.V. PATCH by Richard D. Lowe.

### BACKGROUND OF THE INVENTION

1. Field of the Invention

The present invention relates generally to the field of wound dressings, and specifically to a dressing that provides care for each phase of the healing cycle individually with optimal targeted efficiency for use directly on a wound as well as for securing medical devices to a patient's skin, such as intravenous catheters.

2. Description of the Prior Art

Wound dressings typically provide for the coverage of clean, closed surgical incisions, abrasions, skin tears, blisters, skin graft donor sites, first and second degree burns, and stage I or II pressure ulcers. Additionally, adhesive wound dressings can be used to secure medical equipment to a patient's skin, such as over catheter sites and devices, including peripheral and midline venous catheters, central venous catheters, which include subclavian, jugular, femoral and PICCs, pulmonary artery catheters, arterial catheters, dialysis catheters, implanted infusion devices (ports), epidural catheters, insulin pump catheters, scalp vein needles and feeding tubes.

Heretofore, there has not existed a single product that could be used as both an improved two-step wound dressing as well as a securing device for medical equipment to be adhered to a patient's skin that also included improved healing properties.

During the healing cycle, cells required for angiogenesis (i.e., the generation of new blood vessels) require a moist environment as angiogenesis occurs towards regions with a deficiency of oxygen in the tissues (hypoxia). In the final stage of epithelialization, contracture occurs as the keratinocytes (squamous cells) differentiate to form the protective outermost layer of skin cells (stratum corneum). Any restriction of natural oxygen/moisture transference at this stage can cause homeostasis disruption due to accelerated apoptosis of the stratum corneum, not only impeding the body's natural rejuvenation process, but resulting in discomfort due to inflammation and itching.

Typically, in the past, a single ply wound dressing was used to cover wounds. A single ply wound dressing, however, offers only one moisture vapor/oxygen transmission rate to accommodate all stages of the healing cycle, approximately 800 g/m²/24 hr moisture vapor transmission rate and approximately 14,275 cc/m²/24 hr. Traditionally, tape and gauze dressings have been used for the applications for which the instant invention provides a solution. But unlike conventional tape and gauze dressings, the instant invention provides good edge seals, creating a perimeter barrier against peripheral migration of microorganisms. There exists a need for a device that can specifically achieve a more effective occlusive wound dressing helping to create the hypoxic environment necessary for angiogenesis/cell rejuvenation while providing a barrier to cease the migration of microorganisms to the wound.

### SUMMARY OF THE INVENTION

The preferred embodiment of the present invention teaches a wound dressing comprising a base layer having a perimeter portion, a center portion, a top side and a bottom side, the bottom side having adhesive applied thereon; a plurality of apertures located in the center portion of the base layer; a top layer having a perimeter portion, a center portion, a top side and a bottom side, the bottom side having adhesive applied thereon in the perimeter portion of the bottom side; and a pull tab attached to the perimeter portion of the top layer for easy removal of the top layer from the bottom layer.

The above embodiment can be further modified by defining that the wound dressing is made of a pliable polymer.

The above embodiment can be further modified by defining that the wound dressing is transparent.

The above embodiment can be further modified by defining that the adhesive is a non-aggressive adhesive appropriate for medical use.

The above embodiment can be further modified by defining that the bottom layer has a 100% moisture vapor transmission rate and oxygen transfer rate due to vent holes for the final stage of healing.

The above embodiment can be further modified by defining that the top layer has a moisture-vapor transmission rate of 9.4 g/m²/24 hrs and an oxygen transmission rate 2950 g/m²/24 hrs.

An alternative embodiment of the instant invention defines a procedure for dressing a wound comprising the steps of applying a wound dressing to a wound, the wound dressing further comprising a base layer having a perimeter portion, a center portion, a top side and a bottom side, the bottom side having adhesive applied thereon; a plurality of apertures located in the center portion of the base layer; a top layer having a perimeter portion, a center portion, a top side and a bottom side, the bottom side having adhesive applied thereon in the perimeter portion of the bottom side; and a pull tab attached to the perimeter portion of the top layer for easy removal of the top layer from the bottom layer; once rejuvenated dermis is observed, removing the top layer from the wound dressing.

The above embodiment can be further modified by defining that the wound dressing is made of a pliable polymer.

The above embodiment can be further modified by defining that the wound dressing is transparent.

The above embodiment can be further modified by defining that the adhesive is a non-aggressive adhesive appropriate for medical use.

The above embodiment can be further modified by defining that the bottom layer has a 100% moisture vapor transmission rate and oxygen transfer rate due to vent holes for the final stage of healing.

The above embodiment can be further modified by defining that the top layer has a moisture-vapor transmission rate of 9.4 g/m²/24 hrs and an oxygen transmission rate 2950 g/m²/24 hrs.

A second alternative embodiment of the instant invention defines a procedure for securing a medical instrument to the skin of a patient comprising the steps of applying a wound dressing to the patient's skin, the wound dressing further comprising a base layer having a perimeter portion, a center portion, a top side and a bottom side, the bottom side having adhesive applied thereon; a plurality of apertures located in the center portion of the base layer; a top layer having a perimeter portion, a center portion, a top side and a bottom side, the bottom side having adhesive applied thereon in the perimeter portion of the bottom side; and a pull tab attached to the perimeter portion of the top layer for easy removal of the top layer from the bottom layer; removing the top layer from said wound dressing.

The above embodiment can be further modified by defining that the wound dressing is made of a pliable polymer.

The above embodiment can be further modified by defining that the wound dressing is transparent.

The above embodiment can be further modified by defining that the adhesive is a non-aggressive adhesive appropriate for medical use.

The above embodiment can be further modified by defining that the bottom layer has a 100% moisture vapor transmission rate and oxygen transfer rate due to vent holes for the final stage of healing.

The above embodiment can be further modified by defining that the top layer has a moisture-vapor transmission rate of 9.4 g/m²/24 hrs and an oxygen transmission rate 2950 g/m²/24 hrs.

### BRIEF DESCRIPTION OF THE DRAWINGS

FIG. 1 is a perspective view of the invention before it is applied to a wound.

FIG. 2 is a perspective view of the invention as it is applied to a wound.

FIG. 3 is a perspective view of the invention with the top layer being removed.

FIG. 4 is a perspective view of the invention with the top layer removed.

FIG. 5 is an exploded view of both layers of the invention.

FIG. 6 is a side view of the invention.

### DETAILED DESCRIPTION OF A PREFERRED EMBODIMENT

Turning to the drawings, the preferred embodiment is illustrated and described by reference characters that denote similar elements throughout the several views of the instant invention.

The wound aid system 5 offers a two step dressing procedure, with each step designed specifically to address each phase of the healing cycle individually, without compromise and with optimal targeted efficiency. During the healing cycle, cells required for angiogenesis, i.e., proliferation of new blood vessels, require a moist environment as angiogenesis occurs toward regions of low oxygen tension (hypoxia). In the final stage of epithelialization, contracture occurs as the keratinocytes (squamous cells) differentiate to form the protective outermost layer of skin cells (stratum corneum). Any restriction of natural oxygen/moisture transference at this stage can cause homeostasis disruption due to accelerated apoptosis of the stratum corneum, not only impeding the body's natural rejuvenation process, but resulting in discomfort due to inflammation and itching.

As opposed to a single ply wound dressing, which offers only one moisture vapor/oxygen transmission rate to accommodate all stages of the healing cycle, i.e., MVTR (moisture-vapor transmission rate) being approximately 800 g/sq²/24 hrs and OTR (oxygen transmission rate) being approximately 14,275 cc/sq²/24 hrs, the wound dressing's top layer 10a is formulated for a lower moisture vapor/oxygen transmission (MVTR 9.4 g/sq²/24 hrs—OTR 2950 g/sq²/24 hrs). These parameters are chosen specifically to achieve a more effective occlusive wound dressing, thereby helping to create the hypoxic environment necessary for angiogenesis/cell rejuvenation while simultaneously providing a barrier to cease the migration of microorganisms into the wound. In this first phase of the healing cycle, the wound dressing's 10 top layer 10a should remain in place until rejuvenated epidermis is evident.

Once rejuvenated epidermis is evident, the top layer 10a becomes counter productive and is then removed from the base 10b because angiogenesis is complete at the dermal layer. The base layer 10b will continue to provide good edge seals against the peripheral migration of microorganisms and protect the vulnerable tissue during the continued time needed for stratum corneum rejuvenation and maturation, thereby building tensile strength. Centrally located vent holes 22 allow 100% natural moisture/vapor oxygen transmission to prevent accelerated apoptosis/maceration/impedance during this final stage of cell rejuvenation.

The wound dressing 10 can also be used to secure intravenous devices to a patient's skin. The wound dressing 10 is a polymer intravenous dressing that brings together the best of both occlusive and conventional dressing methods in one convenient, comfortable and cost-effective product. Like occlusive dressings, the wound dressing 10 provides a barrier against peripheral migration of microorganisms. Additionally, due to the centrally located vent holes 22, it also provides the same 100% natural ventilation of the conventional tape and gauze method.

Unlike occlusive dressings, the wound dressing 10 provides 100% moisture vapor and oxygen transference for the highest possible ventilation. The high moisture vapor/oxygen transmission prevents the possibility of maceration and/or hyper-degeneration of surface skin cells (stratum corneum), adding to the patient's comfort because it eliminates the possibility of inflammation/itch sensation.

Unlike conventional tape and gauze dressings, the wound dressing 10 provides good edge seals, creating a perimeter barrier against peripheral migration of microorganisms. The wound dressing 10 is specifically designed for the convenience of the healthcare provider with a simple and convenient two step application, which due to its size and shape, does not require additional tape and time. It is simply removed from its liner and then placed over the catheter and IV line. It provides both protection and stabilization while simultaneously allowing free mobility without worry to the patient and health care provider of any disturbance to the intravenous infusion.

The wound dressing 10 is pliable with non-aggressive adhesive 23a, 23b and provides 100% ventilation for absolute comfort during wear time and removal. It is transparent, providing for 100% visualization and observation. It is cost effective, requiring no additional tape or time. It is convenient and secure for both the patient and the health care provider.

The wound dressing 10 can be used as an autolytic debridement facilitated by the moist wound healing environment. It can be used as a secondary dressing over gauze, alignates or hydrogels. It can be used as a protective eye covering. It can also provide skin protection against moisture and friction.

The wound dressing 10 is typically constructed of two 3″×4.5″ polyethylene oval bands 10a, 10b. The base 10b is made from 3M® 1525L and is 3.0 mil thick. With a centered oval pattern that is 2″×2.75″ of 36 3/16″ circular vent holes 22, providing for a ½″ wide perimeter of solid base around the vent holes 22. There is 100% adhesive coverage applied on the bottom of the base 10b for maximum skin adhesion, typically employing a 3M® non-aggressive/bio-compatible adhesive.

The top layer 10a is 3M® 9719, which has a very low MVTR of 9.4 g/sq²/24 hrs for occlusivity, and is 100% solid, i.e., there are no vent holes. It is 1.7 mil thick. The top layer 10a has partial adhesive coverage 23a applied on bottom. There is a voided area with no vent holes that is 3.0 mil thick. There is a ¼″ oval pull tab 20 with no adhesive at one end, allowing for a ½″ thick perimeter of adhesive around the voided area, thereby allowing an easy peel from the base 10b when necessary. Dow Corning® MG-0560 bio compatible adhesive is typically used.

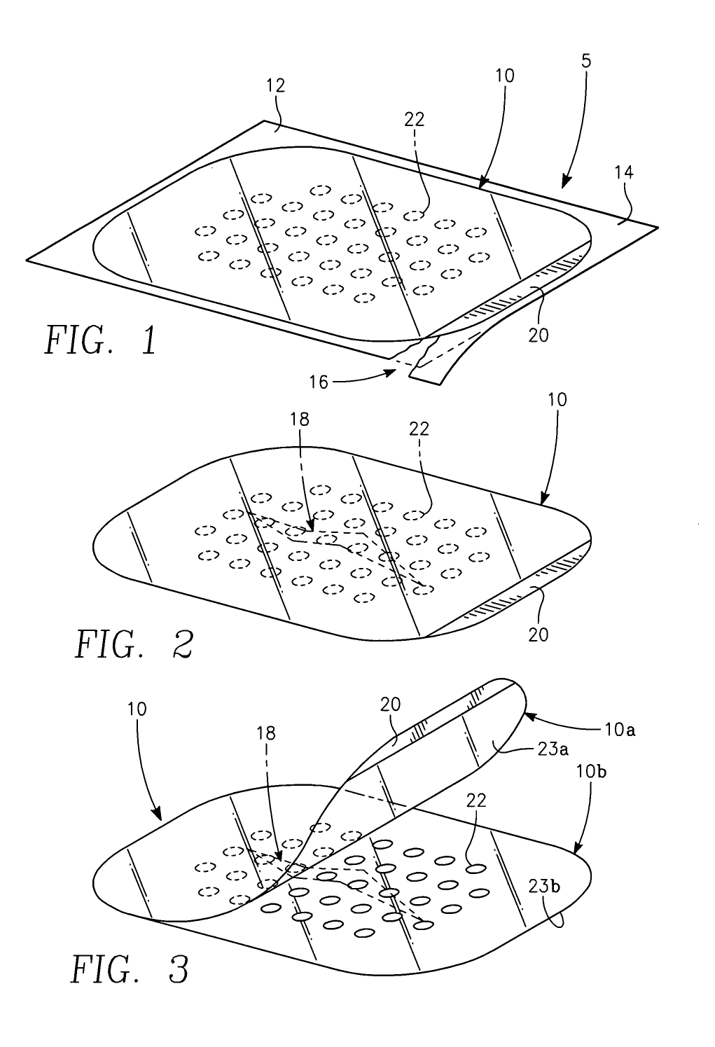

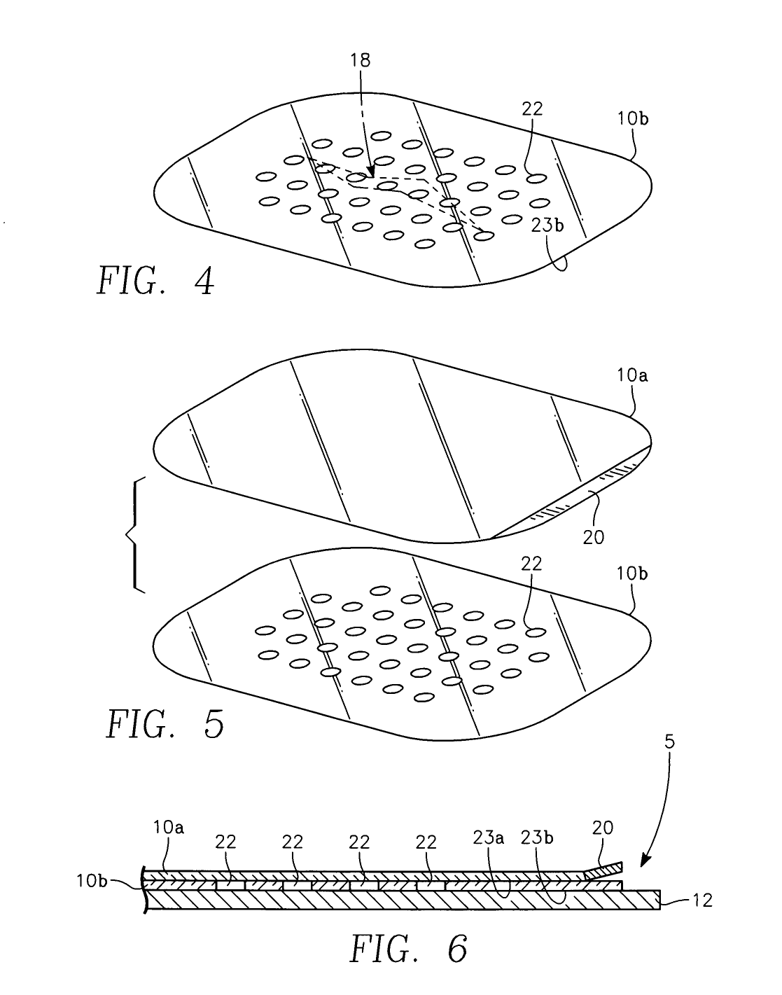

As seen in FIG. 1, the wound aid system 5 is a two piece wound cover 10. The top layer 10a is the low moisture/vapor layer. The base layer 10b allows for the natural evaporation of moisture due the vent holes 22. The two piece wound cover 10 comes attached to a backing 12. There is a cut 14 on each side of the backing 12 to allow for removal therefrom.

In FIG. 1, it can be seen where the cut 14 is torn 16 to allow removal of the base layer 10b from the backing 12. The base layer 10b is then applied over the wound 18. The top layer 10a is removed from the base layer 10b through the pull tab 20.

As seen in FIG. 6, there is are adhesive layers 23a, 23b between the top layer 10a and the base layer 10b as well as between the base layer 10b and the backing 12.

The illustrations and examples provided herein are for explanatory purposes and are not intended to limit the scope of the appended claims. This disclosure is to be considered an exemplification of the principles of the invention and is not intended to limit the spirit and scope of the invention and/or claims of the embodiment illustrated. Those skilled in the art will make modifications to the invention for particular applications of the invention.

What is claimed is:

1. A wound dressing comprising
a base layer having a perimeter portion, a center portion, a top side and a bottom side, said bottom side having adhesive applied thereon;
a plurality of apertures located in said center portion of said base layer;
a top layer having a perimeter portion, a center portion, a top side and a bottom side, said bottom side having adhesive applied thereon in said perimeter portion of said bottom side; and
a pull tab attached to the perimeter portion of said top layer for easy removal of said top layer from said bottom layer.

2. A wound dressing as defined in claim 1 wherein said wound dressing is made of a pliable polymer.

3. A wound dressing as defined in claim 1 wherein said wound dressing is transparent.

4. A wound dressing as defined in claim 1 wherein said adhesive is a non-aggressive adhesive appropriate for medical use.

5. A wound dressing as defined in claim 1 wherein said bottom layer has a moisture-vapor transmission rate and oxygen transfer rate due to vent holes for the final stage of healing.

6. A wound dressing as defined in claim 1 wherein said top layer has a moisture-vapor transmission rate of 9.4 g/m²/24 hrs and an oxygen transmission rate 2950 g/m²/24 hrs.

7. A procedure for dressing a wound comprising the steps of
applying a wound dressing to a wound, said wound dressing further comprising
a base layer having a perimeter portion, a center portion, a top side and a bottom side, said bottom side having adhesive applied thereon;
a plurality of apertures located in said center portion of said base layer;
a top layer having a perimeter portion, a center portion, a top side and a bottom side, said bottom side having adhesive applied thereon in said perimeter portion of said bottom side; and
a pull tab attached to the perimeter portion of said top layer for easy removal of said top layer from said bottom layer;
once rejuvenated dermis is observed, removing said top layer from said wound dressing.

8. A procedure as defined in claim 7 wherein said wound dressing is made of a pliable polymer.

9. A procedure as defined in claim 7 wherein said wound dressing is transparent.

10. A procedure as defined in claim 7 wherein said adhesive is a non-aggressive adhesive appropriate for medical use.

11. A procedure as defined in claim 7 wherein said bottom layer has a moisture-vapor transmission rate and oxygen transfer rate due to vent holes for the final stage of healing.

12. A procedure as defined in claim 7 wherein said top layer has a moisture-vapor transmission rate of 9.4 g/m²/24 hrs and an oxygen transmission rate 2950 g/m²/24 hrs.

13. A procedure for securing a medical instrument to the skin of a patient comprising the steps of
applying a wound dressing to said patient's skin, said wound dressing further comprising
a base layer having a perimeter portion, a center portion, a top side and a bottom side, said bottom side having adhesive applied thereon;
a plurality of apertures located in said center portion of said base layer;
a top layer having a perimeter portion, a center portion, a top side and a bottom side, said bottom side having adhesive applied thereon in said perimeter portion of said bottom side; and
a pull tab attached to the perimeter portion of said top layer for easy removal of said top layer from said bottom layer;
removing said top layer from said wound dressing.

14. A procedure as defined in claim 13 wherein said wound dressing is made of a pliable polymer.

15. A procedure as defined in claim 13 wherein said wound dressing is transparent.

16. A procedure as defined in claim 13 wherein said adhesive is a non-aggressive adhesive appropriate for medical use.

17. A procedure as defined in claim 13 wherein said bottom layer has a moisture-vapor transmission rate and oxygen transfer rate due to vent holes for the final stage of healing.

18. A procedure as defined in claim 13 wherein said top layer has a moisture-vapor transmission rate of 9.4 g/m²/24 hrs and an oxygen transmission rate 2950 g/m²/24 hrs.
